(19) United States
(12) Patent Application Publication
(10) Pub. No.: US 2015/0313941 A1
(43) Pub. Date: Nov. 5, 2015

(54) Composition and methods for amelioration of diseases and related symptoms in domestic animals

(71) Applicant: Jeffrey Alan McKinney, Lafayette, CA (US)

(72) Inventor: Jeffrey Alan McKinney, Lafayette, CA (US)

(21) Appl. No.: 14/545,289

(22) Filed: Apr. 17, 2015

Related U.S. Application Data
(60) Provisional application No. 61/996,223, filed on Apr. 30, 2014.

(51) Int. Cl. A61K 33/30; A61K 31/375; A61K 31/355; A61K 36/45; A61K 31/047; A61K 36/07; A61K 31/385; A61K 36/752; A61K 36/185; A61K 36/22; A61K 36/87; A61K 36/73; A61K 31/07; A61K 31/353

(52) U.S. Cl. CPC A61K 33/30; A61K 31/07; A61K 31/375; A61K 31/355; A61K 36/45; A61K 31/047; A61K 31/353; A61K 31/385; A61K 36/752; A61K 36/185; A61K 36/22; A61K 36/87; A61K 36/73; A61K 36/07

(57) ABSTRACT

The present invention is generally directed to compositions and methods for amelioration of diseases and related symptoms in domestic animals. In one aspect, the invention is directed to a method of ameliorating the symptoms of arthritis, brain aging, cataracts, diabetes, dermatitis, FLUTD, glaucoma, low energy levels or obesity in a domestic animal, wherein the method comprises administering a composition to the animal, wherein the composition comprises a Fruit-Based Additive, a Vegetable-Based Additive or a Fruit- and Vegetable-Based Additive.

### FIELD OF THE INVENTION

The present invention is generally directed to compositions and methods for amelioration of diseases and related symptoms in domestic animals.

### BACKGROUND OF THE INVENTION

There have been reports regarding the treatment of diseases and related symptoms in domestic animals. For example, U.S. Pat. No. 7,414,019, issued Aug. 19, 2008, is allegedly directed to “a method for treating a bone condition in a patient, e.g., a mammal, a human, a horse, a dog, or a cat. The method includes administering an effective amount of FGF-8, FGF-8 analog, or a FGF-8 agonist to the patient.” Abstract.

U.S. Pat. No. 7,208,579, issued Apr. 24, 2007, is allegedly directed to “provid[ing] [a] horse, cat and dog IL4 receptor, including soluble receptors as well as method for therapeutic use of such receptors.” Abstract.

U.S. Pat. No. 6,875,584, issued Apr. 5, 2005, is allegedly directed to: “Polypeptide and polynucleotide vaccines effective to treat or prevent infection of a mammal, such as a dog, a cat, or a human, by a protozoan. Methods of treatment and prevention are also provided, including therapeutic administration of the vaccine to an infected mammal to prevent progression of infection to a chronic debilitating disease state. Preferred embodiments of the polynucleotide vaccine contain nucleotide coding regions that encode polypeptides that are surface-associated or secreted by T. cruzi. Optionally the efficacy of the polynucleotide vaccine is increased by inclusion of a nucleotide coding region encoding a cytokine. Preferred embodiments of the polypeptide vaccine include immunogenic peptides that contain membrane transducing sequences that allow the polypeptides to translocate across a mammalian cell membrane.” Abstract.

Despite various reports, there is still a need for new compositions and methods for amelioration of diseases and related symptoms in domestic animals.

### SUMMARY OF THE INVENTION

In one aspect, the invention is directed to a method of ameliorating the symptoms of arthritis, brain aging, cataracts, diabetes, dermatitis, FLUTD, glaucoma, low energy levels or obesity in a domestic animal, wherein the method comprises administering a composition to the animal, wherein the composition comprises a Fruit-Based Additive, a Vegetable-Based Additive or a Fruit- and Vegetable-Based Additive.

### DETAILED DESCRIPTION OF THE INVENTION

The present invention is generally directed to compositions and methods for amelioration of diseases and related symptoms in domestic animals.

“Cesium Salt” refers to a compound that includes a positively charged cesium ion and a negatively charged counterion. Nonlimiting examples of such compounds include: cesium arginate, cesium ascorbate, cesium bicarbonate, cesium caprylate, cesium chloride, cesium cysteinate, cesium citrate, cesium fumarate, cesium humate, cesium fulvate, cesium methionate, cesium glutamate, cesium gluconate, cesium glycinate, cesium aspartate, cesium lysinate, cesium succinate, cesium carbonate, cesium lactate, cesium malate, cesium tartrate, cesium sulfate, cesium phosphate, cesium nitrate, cesium fluoride, cesium bromide, cesium iodide, cesium orotate, and mixtures thereof.

“Dehydrated Domestic Animal Food” refers to food materials suitable for domestic animal feeding that have been dehydrated and which can be reconstituting upon mixing with one or more liquids. The food can include a single ingredient (e.g., chicken or fish) or multiple ingredients. The ingredients can be raw or cooked. Nonlimiting examples of ingredients include: chicken, fish, beef, lamb, venison, eggs, potatoes, sweet potatoes, flaxseed, pumpkin, spinach, cranberries, and mixtures thereof.

“Domestic Animal” refers to any of various animals that have been tamed and made fit for a human environment. Nonlimiting examples of Domestic Animals include: cats, dogs and horses.

“Freeze Drying” refers to preserving something by rapidly freezing it and then subjecting it to a high vacuum that removes ice by sublimation. A preferred freeze drying process includes at least the following steps: macerating fruits and/or vegetables; rinsing the macerated fruits and/or vegetables with chilled water (e.g., 45° C.); macerating the fruits and/or vegetables again; after stabilizing and blending the macerated fruits and/or vegetables, subjecting the mixture to a chill tunnel (e.g., 45° C. to 50° C.); and freeze drying the mixture (e.g., vacuum at approximately −40° C.).

“Fruit-Based Additive” refers to a fruit extract or concentrate. The extract or concentrate includes material from a single or from multiple fruits. One method of making a Fruit-Based Additive concentrate is freeze drying. Nonlimiting examples of fruits that are used to make the additive include dried forms of the following: tomato, banana, cranberry, apple, orange, pomegranate, grape, strawberry, papaya, mango, grape, shitake mushroom and mixtures thereof. In one case, the additive includes the following fruits: orange, papaya/mango, cranberry, grape, strawberry, and shitake mushroom. In another case, the additive includes the following fruits: orange, papaya/mango, cranberry, grape, strawberry, pomegranate, and shitake mushroom.

“Fruit- and Vegetable-Based Additive” refers to a fruit and vegetable extract or concentrate. The extract or concentrate includes material from a single fruit and vegetable or from multiple fruits and vegetables. One method of making a Fruit- and Vegetable-Based Additive concentrate is freeze drying. Nonlimiting examples of fruits and vegetables that are used to make the additive include dried forms of the following: tomato, cranberry, apple, orange, pomegranate, grape, strawberry, papaya, mango, banana, grape, broccoli, carrot, shitake mushrooms, spinach, beet, and mixtures thereof. In one case, the additive includes the following fruits and vegetables: tomato, broccoli, carrot, shitake mushrooms, cranberry, apple, and orange. In another case, the additive includes the following fruits and vegetables: cranberry, pomegranate, orange, grape, strawberry, and shitake mushrooms. In still another case, the additive includes the following fruits and vegetables: spinach, broccoli, carrot, tomato, beet, and shitake mushrooms. In another case, the additive includes the following fruits and vegetables: spinach, broccoli, carrot, beet, tomato, shitake mushroom, apple, cranberry, pomegranate, orange, grape and strawberry. In another case, the additive includes the following fruits and vegetables: spinach, broccoli, carrot, beet, tomato, shitake mushroom, apple, cranberry, pomegranate, orange, grape, and strawberry. In another case, the additive includes the following fruits and vegetables: tomato, broccoli, carrot, shitake mushroom, apple, orange, and banana.

“Rubidium Salt” refers to a compound that includes a positively charged rubidium ion and a negatively charged counterion. Nonlimiting examples of such compounds include: rubidium arginate, rubidium ascorbate, rubidium bicarbonate, rubidium caprylate, rubidium fulvate, rubidium methionate, rubidium glutamate, rubidium gluconate, rubidium glycinate, rubidium aspartate, rubidium lysinate, rubidium succinate, rubidium carbonate, rubidium lactate, rubidium malate, rubidium tartrate, rubidium sulfate, rubidium phosphate, rubidium nitrate, rubidium fluoride, rubidium bromide, rubidium iodide, rubidium orotate, and mixtures thereof.

“Symptoms of Arthritis” refers to pain and inflammation in the joints of a domestic animal, as well as soft tissue thickening, narrowing of joint spaces, joint effusion (fluid build-up), cartilage changes, bone changes, mineralization of soft tissues, intra-articular calcified bodies (osteophytes) and other physical changes that are known to be associated with arthritis. Radiographs, commonly called X-rays; are a very effective tool for identifying and assessing arthritis.

“Arthritis Nutritional” refers to one or more of the following: chondroitin sulfate; glucosamine sulfate; calcium; vitamin D3; ginger, turmeric; omega-3; green tea; SAMe; MSM; stinging nettle; bromelian; vitamin E; and, Devil's claw.

“Symptoms of Brain Aging” refers to a disturbance of activity and sleep patterns, an increase in aimless wandering and pacing, compulsive behaviors (e.g., circling, tremors, stiffness and weakness), housetraining issues. Brain aging is often diagnosed as the result of a neurological examination, where one or more of the following are assessed: mentation (e.g., level and quality of consciousness); posture (e.g., head tilt, base of stance, decerebrate posture), gait (e.g., paresis, ataxia), postural reactions (e.g., paw positioning, hopping), spinal reflexes (e.g., patellar reflex, withdrawal reflex), cranial nerves (e.g., vision, menace response, pupillary light response), palpation (e.g., swelling or atrophy), nociception (e.g., superficial or deep pain).

“Brain Aging Nutritional” refers to one or more of the following: vitamin B1; vitamin C; vitamin D; vitamin E; beta-carotene; ginseng; green tea extract; and, cod liver oil.

“Symptoms of Cataracts” refers to cloudy, white-ish or blue-grey pupils. Cataracts are oftentimes diagnosed by dilation of pupils and evaluation of cataract extent, the Schirmer tear test, and staining the eye with a fluorescein dye.

“Cataract Nutritional” refers to one or more of the following: vitamin C; Vitamin E; bilberries; beta-carotene; astaxanthin; glutathione; alpha-lipoic acid.

“Symptoms of Conjunctivitis” refers to squinting or spasmodic blinking (blephora), redness of the moist tissues of the eye, discharge from the eye(s), swelling of the moist tissue covering the eyeball, follicle formation. Conjunctivitis is oftentimes diagnosed using one or more of the following tests: fluorescein stain; culture of eye discharge; and, biopsy of conjunctiva cells.

“Conjunctivitis Nutritional” refers to one or more of the following: vitamin A; vitamin C; zinc; eyebright; chamomile; licorice root; and, turmeric.

“Symptoms of Diabetes” refers to frequent urination and excessive thirst. Diabetes is oftentimes diagnosed using one or more of the following tests: a urinalysis; a serum biochemistry profile; a complete blood count (CBC).

“Diabetes Nutritional” refers to one or more of the following: chromium; vitamin E; selenium; acarbose; Feline Diabetes Solution; and, Fiber Forte Felin.

“Symptoms of Dermatitis” refers to itchy red bumps, skin inflammation, blisters, crusts, and hair loss.

“Dermatitis Nutritional” refers to one or more of the following: omega-3 fatty acids (EPA and DHA); omega-6 fatty acids (linoleic acid); vitamin C; vitamin E; vitamin A; zinc; reishi mushrooms; echinacea; astragulus root; and, thymus.

“Symptoms of Feline Lower Urinary Tract Disorder (FLUTD)” refers to frequent trips to the litter box, prolonged squatting and straining during attempts to urinate, blood in the urine and urinating outside of the litter box. FLUTD is oftentimes diagnosed using one or more of the following tests: urinalysis; abdominal x-rays; chemistry panel to assess kidney function; complete blood count (CBC).

“FLUTD Nutritional” refers to one or more of the following: glucosamine; chondroitin; and, cosequin.

“Symptoms of Glaucoma” refers to high pressure within the eye, blinking of the eye, recession of the eyeball into the head, redness of blood vessels in the whites of the eyes, cloudy appearance at the front of the eye, dilated pupils, vision loss. Glaucoma is oftentimes diagnosed according to the elevated pressure within an animal's eyes.

“Glaucoma Nutritional” refers to vitamin A, vitamin C, vitamin E, bilberry extract, zinc, lutein, quercetin, alpha lipoic acid, mixed carotenoids, and grape seed extract.

“Symptoms of Lower Energy Level” refers to eating and/or drinking less than normal, walking with difficulty, lethargy.

“Energy Level Nutritional” refers to one or more of the following: eleuthero; rosemary; spirulina; amalaki; vitamin B; vitamin B12; coQ10; and, PQQ.

“Symptoms of Obesity” refers to weight gain, excess body fat, unwillingness to exercise.

“Obesity Nutritional” refers to fiber, chromium picolinate, essential fatty acids, kelp, lecithin, spirulina, vitamin C, and yerba mate.

“Vegetable-Based Additive” refers to a vegetable extract or concentrate. The extract includes material from a single vegetable or from multiple vegetables. One method of making a Vegetable-Based Additive concentrate is freeze drying. Nonlimiting examples of vegetables that are used to make the additive include dried forms of the following: broccoli, carrot, shitake mushrooms, spinach, beet, and mixtures thereof. In one case, the additive includes the following vegetables: spinach, broccoli, carrot, tomato, beet, and shitake mushroom. In another case, the additive includes the following vegetables: spinach, broccoli, carrot, tomato, beet and shitake mushroom.

The compositions and methods of the present invention are directed to the amelioration of symptoms related to arthritis, brain aging, cataracts, conjunctivitis, diabetes, dermatitis, Feline Lower Urinary Tract Disorder, glaucoma, low energy levels, and obesity in domestic animals.

In one case, compositions according to the present invention comprise a Cesium Salt and/or a Rubidium Salt.

In another case, compositions according to the present invention comprise a Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive.

In another case, compositions according to the present invention comprise a Fruit-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin B1; vitamin C; vitamin D; vitamin E; beta-carotene; ginseng; green tea extract; and, cod liver oil. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin C; Vitamin E; bilberries; beta-carotene; astaxanthin; glutathione; alpha-lipoic acid. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin A; vitamin C; zinc; eyebright; chamomile; licorice root; and, turmeric. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: chromium; vitamin E; selenium; acarbose; Feline Diabetes Solution; and, Fiber Forte Felin. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: glucosamine; chondroitin; and, cosequin. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: eleuthero; rosemary; spirulina; amalaki; vitamin B; vitamin B12; coQ10; and, PQQ. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: fiber, chromium picolinate, essential fatty acids, kelp, lecithin, spirulina, vitamin C, and yerba mate. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin B1; vitamin C; vitamin D; vitamin E; beta-carotene; ginseng; green tea extract; and, cod liver oil. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin C; Vitamin E; bilberries; beta-carotene; astaxanthin; glutathione; alpha-lipoic acid. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin A; vitamin C; zinc; eyebright; chamomile; licorice root; and, turmeric. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: chromium; vitamin E; selenium; acarbose; Feline Diabetes Solution; and, Fiber Forte Felin. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: glucosamine; chondroitin; and, cosequin. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: eleuthero; rosemary; spirulina; amalaki; vitamin B; vitamin B12; coQ10; and, PQQ. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: fiber, chromium picolinate, essential fatty acids, kelp, lecithin, spirulina, vitamin C, and yerba mate. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Fruit- and Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin B1; vitamin C; vitamin D; vitamin E; beta-carotene; ginseng; green tea extract; and, cod liver oil. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin C; Vitamin E; bilberries; beta-carotene; astaxanthin; glutathione; alpha-lipoic acid. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin A; vitamin C; zinc; eyebright; chamomile; licorice root; and, turmeric. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: chromium; vitamin E; selenium; acarbose; Feline Diabetes Solution; and, Fiber Forte Felin. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: glucosamine; chondroitin; and, cosequin. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: eleuthero; rosemary; spirulina; amalaki; vitamin B; vitamin B12; coQ10; and, PQQ. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: fiber, chromium picolinate, essential fatty acids, kelp, lecithin, spirulina, vitamin C, and yerba mate. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In one aspect, the present invention is directed to the amelioration of symptoms related to arthritis in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to brain aging in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: vitamin B1; vitamin C; vitamin D; vitamin E; beta-carotene; ginseng; green tea extract; and, cod liver oil. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin B1; vitamin C; vitamin D; vitamin E; beta-carotene; ginseng; green tea extract; and, cod liver oil. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin B1; vitamin C; vitamin D; vitamin E; beta-carotene; ginseng; green tea extract; and, cod liver oil. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to cataracts in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: vitamin C; Vitamin E; bilberries; beta-carotene; astaxanthin; glutathione; alpha-lipoic acid. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin C; Vitamin E; bilberries; beta-carotene; astaxanthin; glutathione; alpha-lipoic acid. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin C; Vitamin E; bilberries; beta-carotene; astaxanthin; glutathione; alpha-lipoic acid. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to conjunctivitis in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: vitamin A; vitamin C; zinc; eyebright; chamomile; licorice root; and, turmeric. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin A; vitamin C; zinc; eyebright; chamomile; licorice root; and, turmeric. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin A; vitamin C; zinc; eyebright; chamomile; licorice root; and, turmeric. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to diabetes in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: chromium; vitamin E; selenium; acarbose; Feline Diabetes Solution; and, Fiber Forte Felin. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: chromium; vitamin E; selenium; acarbose; Feline Diabetes Solution; and, Fiber Forte Felin. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: chromium; vitamin E; selenium; acarbose; Feline Diabetes Solution; and, Fiber Forte Felin. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to dermatitis in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to Feline Lower Urinary Tract Disorder in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: glucosamine; chondroitin; and, cosequin. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: glucosamine; chondroitin; and, cosequin. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: glucosamine; chondroitin; and, cosequin. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to glaucoma in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to low energy levels in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: eleuthero; rosemary; spirulina; amalaki; vitamin B; vitamin B12; coQ10; and, PQQ. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: eleuthero; rosemary; spirulina; amalaki; vitamin B; vitamin B12; coQ10; and, PQQ. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: eleuthero; rosemary; spirulina; amalaki; vitamin B; vitamin B12; coQ10; and, PQQ. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to obesity in domestic animals. The method comprises administering a composition to the domestic animal, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Domestic Animal Food and a Fruit-Based Additive, Dehydrated Domestic Animal Food and a Vegetable-Based Additive, or Dehydrated Domestic Animal Food and a Fruit- and Vegetable-Based Additive;

A Fruit-Based Additive and one or more additives selected from the following: fiber, chromium picolinate, essential fatty acids, kelp, lecithin, spirulina, vitamin C, and yerba mate. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: fiber, chromium picolinate, essential fatty acids, kelp, lecithin, spirulina, vitamin C, and yerba mate. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: fiber, chromium picolinate, essential fatty acids, kelp, lecithin, spirulina, vitamin C, and yerba mate. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

Where one or more cesium and/or rubidium salts are in the composition used to ameliorate symptoms of a domestic animal, the amount of salt included typically ranges from one milligram to one gram. The included amount is suitably varied to obtain a desired level of symptom reduction.

Where Fruit-, Vegetable- or Fruit- and Vegetable-Based additives are in the composition used to ameliorate symptoms of a domestic animal, the amount of additive included typically ranges from one milligram to ten grams. The included amount is suitably varied to obtain a desired level of symptom reduction.

Where Dehydrated Domestic Animal Food is in the composition used to ameliorate symptoms of a domestic animal, the amount of dehydrated food included typically ranges from 100 milligrams to one kilogram. The included amount is suitably varied to obtain a desired level of symptom reduction.

Where additives other than the above are in the composition used to ameliorate symptoms of a domestic animal, the amount included typically ranges from one milligram to 10 grams. The included amount is suitably varied to obtain a desired level of symptom reduction.

1. A method of ameliorating the symptoms of arthritis, brain aging, cataracts, diabetes, dermatitis, FLUTD, glaucoma, low energy levels or obesity in a domestic animal, wherein the method comprises administering a composition to the animal, wherein the composition comprises a Fruit-Based Additive, a Vegetable-Based Additive or a Fruit- and Vegetable-Based Additive.
